(19) United States
(12) Patent Application Publication
(10) Pub. No.: US 2009/0263521 A1
(43) Pub. Date: Oct. 22, 2009

(54) COMPOSITION FOR THE TREATMENT OF CANCERS AND INHIBITION OF METASTASIS CONTAINING EXTRACTS OR FRACTIONS OF THE MAGNOLIA OBOVATA

(75) Inventors: Byoung-Mog KWON, DAEJEON (KR); Dong Cho HAN, DAEJEON (KR); Hye-Nan KIM, DAEJEON (KR); Young-Min HAN, DAEJEON (KR); Dae-Seop SHIN, Koysan-gan (KR)

Correspondence Address: LUCAS & MERCANTI, LLP, 475 PARK AVENUE SOUTH, 15TH FLOOR, NEW YORK, NY, 10016, US

(73) Assignee: KOREA RESEARCH INSTITUTE OF BIOSCIENCE AND BIOTECHNOLOGY, Daejeon (KR)

(21) Appl. No.: 12/191,770

(22) Filed: Aug. 14, 2008

(30) Foreign Application Priority Data
Apr. 18, 2008 (KR) 10-2008-0036190

(51) Int. Cl. A61K 36/575; A61K 31/05; A61P 35/04

(52) U.S. Cl. 424/777; 424/725; 514/733; 424/778

(57) ABSTRACT

The present invention relates to a composition containing the extracts of Magnolia obovata Thunberg or fractions thereof for the prevention and treatment of cancer and for the inhibition of metastasis. Particularly, the extracts of Magnolia obovata Thunberg fruits and floral buds extracted with water, alcohol or a mixture thereof as a solvent and fractions isolated from the same inhibit metastasis by inhibiting migration of those cells over-expressing PRL-3 which plays an important role in cancer cell growth and migration and at the same time induce apoptosis of cancer cells, so that they can be effectively used as a composition for the prevention and treatment of cancer or a composition for the inhibition of metastasis.

### TECHNICAL FIELD

The present invention relates to a composition for the treatment of cancer and inhibition of metastasis containing the extracts of Magnolia obovata Thunberg or fractions thereof as an active ingredient, more precisely, a composition for the prevention and treatment of cancer or inhibition of metastasis containing the extracts of Magnolia obovata Thunberg extracted from its fruits or floral buds with water, alcohol or the mixture thereof as a solvent and active fractions of the same.

### BACKGROUND ART

Cancer is the most serious disease threatening human lives. In Korea, cancer has been the leading cause of death and In USA, cancer has been the second reason of death following cardiovascular disease. Even after all the researches over years, cancer is still the biggest disaster to human taking millions of lives and causing uncounted economical loss. The reason why it is so hard to conquer cancer that damages human lives so much is that cancer is not only developed by the co-works of various reasons including environmental pollution, exposure on toxic materials, drug overuse and genetic transformation over the advanced age but also developed by transformation of normal cells to cancer cells not by foreign infection but by intracellular changes, so that it is very difficult to distinguish normal cells from cancer cells. Besides, a cancer patient does not feel any symptoms, which makes the prevention and treatment of cancer difficult. The conventional anticancer drugs developed so far have serious side effects by cytotoxicity and have problems of inducing secondary cancer. Therefore, it is still a long way to conquer cancer.

Two major reasons of cancer being the leading cause of death are as follows: First, cancer is caused by genetic transformation that is one of the critical factors regulating life-time. Second, unlike cardiovascular disease whose development mechanism has been well understood and thereby whose signs could be reduced by changing life-style, cancer is still in fog with its reasons and development mechanism unknown, indicating there is no preferred method for the prevention of cancer.

About 25 years ago, oncogene defect was first reported. Since then, genetic information has been important factor for the development of anticancer agents. However, the treatment method of cancer still depends on surgical operation, radio-therapy and chemo-therapy administering approximately 40 kinds of highly cytotoxic anticancer agents. These methods are limited to those patients in early cancer stage or with a specific cancer. So, the increase of death rate of cancer is not stopped.

To be transformed into malignant tumor, cancer cells not only need the processes of cell proliferation and invasion into adjacent cells but also something that overpowered the growth regulation signals. In normal cells, there are both growth stimulating signals and growth inhibiting signals among a pack of signals transmitted to nuclei. However, in cancer cells, the connection between growth inhibiting factors are loosened or destroyed and even signals mediated by potential inhibiting factors are neglected. In many cancer cells, important inhibiting factors induced by tumor suppressor genes are defected or inactivated in fact (Bruce A. J. Ponder, Nature, 411, 336-341, 2001).

According to the results of recent studies, the ultimate target of cell growth factors or cell growth inhibitors is the molecular apparatus, which is so called ‘cell cycle’, in nucleus. Every cancer cell is proliferated fast free from cell cycle regulation. In normal cells, intracellular transmitted signals are all regulated and right stage of cell cycle is determined for each cell. If a cell needs to be divided, cell cycle begins to operate (Gerard I. Evan and Karen H. Vousden., Nature, 411, 342-348, 2001). It is one way of developing anticancer agents to inactivate cell growth factors or to activate cell growth inhibitors. However, despite these two control systems are working, some cells are auto activated or continuously replicated with ignoring cell division stop signal, which turn to cancer cells at last. Severe genetic mutation in cells can be another reason of cancer. Once genetic mutation is induced excessively, cell proliferation/differentiation cannot be suppressed with all the defense mechanisms, leading to the development of cancer.

Each human cell is auto-destroyed when its basic component is damaged or cannot be regulated anymore, which is called apoptosis. For examples if a gene in the nucleus is damaged, it leads to apoptosis. According to recent studies, the expression of an oncogene or malfunctioning of a tumor suppressor gene also induces apoptosis. The destruction of a damaged cell itself is not good for the cell but good for the entire body. Because, the destruction of the cell showing genetic mutation is advantages in eliminating potential risk. But, not often though, abnormal apoptosis rather causes cancer from the cell having a mutant gene (Catherine Denicourt and Steven F. Dowdy, Science, 305, 1411-1413, 2004).

Cancer cells in progress have their way to avoid apoptosis. The tumor suppressor protein p53 can destroy cells if necessary. But, once this protein is inactivated, auto-destruction ability of the cell is reduced. In addition, cancer cells avoid apoptosis by mass-producing Bcl-2 which is the protein interrupts apoptosis. Scientists have recently disclosed that the factor interrupting apoptosis is not only a reason of tumor spread but also a cause of tumor cell resistance against anti cancer agents, increasing risks in cancer patients. It has been believed over decades that radio-therapy and chemo-therapy using a variety of chemicals kill malignant tumor cells by destroying a large area harboring the genes of tumor cells. However, cancer cells could avoid apoptosis and might not sensitively response to anticancer agents. Therefore, a novel radio-therapy and chemo-therapy specifically targeting apoptosis of cancer cells can be effectively used to conquer cancer (John C. Reed, Nature Review Drug Discovery 1, 111-121, 2002).

Every type of cancer is spread sooner or later, more precisely every primary tumor mass infiltrates into adjacent tissues and anchors in there to proliferate. This metastasis takes 90% of the death rate of cancer. Owing to the capability of infiltration and metastasis, cancer cells can migrate and be growing in a new place where oxygen and nutrition are sufficient in the early stage. Newly generated tumor mass is composed of cancer cells and normal cells recruited from host tissues as an assistant. Infiltration and metastasis are very complicated processes and their genetic-biochemical mechanisms are not disclosed yet.

Metastasis is mainly divided into lymphatic metastasis and hematogenous metastasis. Migration to a far area from the primary region is allegedly due to hematogenous metastasis. Hematogenous metastasis requires multiple step process, for example cell adhesion with endothelial cells or platelets in a specific organ, invasion to adjacent tissues, angiogenesis and cell migration, etc.

That is, metastasis is composed of three main steps, cell adhesion, invasion and angiogenesis. So, if one of these three steps can be inhibited, metastasis is expected to be inhibited. To spread cancer cells to the other tissues, cell migration is necessary. The report regarding metastasis related genes by K. Kinszler and B. Vogelstein team of Johns Hopkins Medical Institution (Science, 2001) encouraged many scientists specialized in metastasis. They found out that PTPase (protein-tyrosine phosphatase) was over-expressed in colon cancer cells transferred into the liver by SAGE (serial analysis of gene expression) and real time PCR. They suggested that PRL-3 gene was directly related to metastasis by confirming that the expression of PTPase was very low in comparatively non-metastatic tumor and normal epithelial tissue. This result paved a clear way to the goal of inhibiting metastasis and established a very effective direct index for diagnosing cancer and for the development of a therapeutic agent for metastasis as well (S. Saha, et al., Science, 294, 1343, 2001).

Oxygen and nutrition supply through vascular network is essential for the survival and normal functions of cells. Every cell in tissue exists within 100 μm from capillary vessel. Once a new tissue is generated, angiogenesis and growth are on stand-by and controlled elaborately. Considering dependence on the adjacent capillary vessel, cells proliferating in tissues have to have endogenous capability to induce angiogenesis. Cells causing a disease by abnormal proliferation are deficient in angiogenesis inducing capability at first, suggesting Lack of powder of spreading. But, as cells are growing, different signals are balanced to promote or inhibit angiogenesis. One of the signals is transmitted by a soluble factor and a receptor in endothelial cell membrane and plays an important role by linking intergrin to adherent molecules, cell-substrate or cell-cell. Signals related to angiogenesis are exemplified by VEGF and FGF1/2. Each of them binds to tyrosine kinase receptor in endothelial cell to transmit a signal to nucleus to form a new blood vessel. Up to date, at least 20 angiogenesis inducers or similar numbers of inhibitor proteins have been known.

Fast growing cancer cells break the balance between angiogenesis inducers and inhibitors to activate an angiogenesis switch. The general way to break the balance is to change gene transcription. In tumor cells, the expression of VEGF or FGF is increased, compared with that in normal cells. On the contrary, the expression of thrombospondin-1 or β-interferon is reduced in tumor cells. For metastasis and growth of tumor cells, angiogenesis is essential, and therefore an angiogenesis inhibitor can be a worthy candidate for an anticancer agent. Such metastasis inhibitors not only are effective in animal tests but also provide a solution for multi-drug resistance that has long been a problem of the conventional chemo-therapy (Massimo Cristofanilli, Chusilp Charnsangavej and Gabriel N. Hortobagyi, Nature Review Drug Discovery, 1, 415, 2002).

There are three kinds of silver magnolia: Magnolia obovata Thunberg, Magnolia officinale Rehder et Wils and Machilus thunbergii Sieb. et Zucc. Magnolia obovata Thunberg belongs to Magnolia family and is originated from Japan, which is cultivated in the south of the central region of Korea. The bark of this tree is called magnoliae cortex because the xylem is monotonous and bark is thick. The tree and its barks are taken off and dried to be used as a medicinal herb. Magnolia obovata Thunberg contains approximately 1% essential oil whose major components are machiol, the sesquiterpene (β-eudesmol), γ-eudesmol, magnolol and honokiol. It also contains alkaloids such as 1-magnocurarine, magnoflorine, anonine, liriodenine, michelarbine and salicifoline. The bark of Magnolia officinale Rehder et Wils is in board or half-tubular shape, which is 2-7 mm in thickness. The outer skin is coarse and light ash or light brown. Sometimes it is reddish brown when cork layer is peeled off. The inner skin is light brown or dark purplish brown and its section is fibered having light reddish brown or purplish brown. This tree contains magnolol, isomagnolol, honokiol and machiol as major components. Machilus thunbergii Sieb. et Zucc, distributed in Korea, Japan, Taiwan and south China, is an evergreen tree belonging to Magnoliopsida, Ranunculales, Lauraceae which is growing on a mountainous district. This tree is 20 m in height and 1 m in diameter. Leaves are crossed each other and in the shape of upside down egg like oval or rather long oval without hairs. It blossoms in May-June and its flowers are yellow green and androgynous. The fruit of this tree is round and 1 cm in diameter and it ripens blackish purple and the fruit stalk is red. This tree contains magnolol, isomagnolol, honokiol and machiol as major components. It has been known that the silver magnolia has the effects of relaxing striated muscle, preventing and treating stomach ulcer, relieving duodenal convulsion, inhibiting gastric juice secretion, suppressing CNS, lowering blood pressure, inhibiting intestinal or bronchial smooth muscle and anti-microbial activity (Ahn Duk Kyun, Illustrated Book of Korean Medicinal Herb, Koyhak Publishing Co., Ltd., p 468, 2000; Kim Chang Min et al., Illustrated book of oriental medicine, Academy Book Co., Ltd., p 199, 2001; Doosan Rncyber; Illustrated book of Korean floa, Koyhak Publishing Co., Ltd., 1998; Illustrated book of Korean medicinal herbs, Koyhak Publishing Co., Ltd., 1998).

In relation to anticancer effect of silver magnolia, the reports have been made by Korean Patent Application No. 2006-0068803 describing that the extract of silver magnolia bark inhibits MMPs and Korean Patent No. 0697236 describing that obovatol isolated from the extract of silver magnolia leaf has anticancer activity by inhibiting cancer cell growth and MMPs. However, there has been no report on the anticancer effect of fruit or floral bud extract of Magnolia obovata Thunberg or fractions thereof. Only honokiol and magnolol were isolated from the above extract of silver magnolia bark and there have not been reports on extracts or fractions containing all three major components, honokiol, obovatol and magnolol, which could be isolated by the present inventors in this invention from the fruits and floral buds of Magnolia obovata Thunberg.

Therefore, the present inventors confirmed that the extracts of Magnolia obovata fruits and floral buds which have long been used as a medicinal herb and the partially purified mixture thereof had the effect of inhibiting metastasis and inducing apoptosis of a cancer cell by inhibiting migration of PRL-3 over-expressing cells which play an important role in tumor cell growth and migration. The present inventors further completed this invention by confirming the anticancer effect of the extracts of Magnolia obovata fruits, and floral buds or fractions thereof in animal tests.

### DISCLOSURE

### Technical Problem

It is an object of the present invention to provide a composition for the prevention and treatment of cancer, a composition for the inhibition of metastasis and a health improving functional food for the prevention and improvement of cancer, all containing the extracts of Magnolia obovata fruits or floral buds and active fractions thereof as an active ingredient.

### Technical Solution

To achieve the above object, the present invention provides a composition for the prevention and treatment of cancer or a composition for the inhibition of metastasis containing the extracts of Magnolia obovata as an active ingredient.

The present invention also provides a composition for the prevention and treatment of cancer or a composition for the inhibition of metastasis containing active fractions of the extracts of Magnolia obovata isolated by the processes of absorbing the extracts on silica gel column and applying hexane and ethyl acetate thereto at the ratio of 8:2 for 20-30 minutes as an active ingredient.

The present invention further provides a method for treating cancer containing the step of administering a pharmaceutically effective dose of the extracts of Magnolia obovata or fractions thereof to a subject with cancer.

The present invention further provides a method for inhibiting metastasis containing the step of administering a pharmaceutically effective dose of the extracts of Magnolia obovata or fractions thereof to a subject with cancer.

The present invention further provides a method for preventing cancer containing the step of administering a pharmaceutically effective dose of the extracts of Magnolia obovata or fractions thereof to a subject with cancer.

The present invention also provides a use of the extracts of Magnolia obovata or fractions thereof for the preparation of a composition for the prevention and treatment of cancer or a composition for the inhibition of metastasis.

The present invention also provides a health improving functional food for the prevention and improvement of cancer containing the extracts of Magnolia obovata as an active ingredient.

The present invention also provides a health improving functional food for the prevention and improvement of cancer containing active fractions of the extracts of Magnolia obovata isolated by the processes of absorbing the extracts on silica gel column and applying hexane and ethyl acetate thereto at the ratio of 8:2 for 20-30 minutes as an active ingredient.

In addition, the present invention provides a use of the extracts of Magnolia obovata or fractions thereof for the preparation of a health improving functional food for the prevention and improvement of cancer.

Hereinafter, terms used in this invention are described.

“Anticancer” herein indicates the action of inhibiting formation or proliferation of cancer cells or inducing apoptosis of tumor cells. In a broad sense, it includes every action for the prevention and treatment of cancer.

“Prevention” herein indicates every action that inhibits tumor formation or delays tumor development by administering the composition of the present invention.

“Treatment” or “improvement” herein indicates every action that relieves symptoms of the said disease or that is advantageous for the treatment of the same disease.

“Administration” herein indicates the action of providing a preferable amount of the composition of the present invention to a patient according to a proper method.

“Patient” herein indicates human and every animal including monkey, dog, goat, pig and rat which have disease that can be improved by the administration of the composition of the present invention.

“Pharmaceutically effective dose” herein indicates the amount of the composition of the present invention which is enough to treat disease and formulated according to reasonable receiving ratio or risk ratio for clinical application. This amount can be determined considering various factors such as kind of a disease, severity of a disease, activity of a drug, sensitivity to a drug, administration time and pathway, elimination rate, term of treatment, drugs co-used, and other factors well-known to those in medical field.

Hereinafter, the present invention is described in detail.

The present invention provides a composition for the prevention and treatment of cancer or a composition for the inhibition of metastasis containing the extracts of Magnolia obovata or active fractions thereof as an active ingredient.

The Magnolia obovata herein is preferably Magnolia obovata Thunberg, but not always limited thereto.

The extracts of Magnolia obovata and active fractions thereof are preferably isolated and purified from the fruits or floral buds of Magnolia obovata, but not always limited thereto.

The extracts of Magnolia obovata and active fractions thereof preferably contain all of obovatol, honokiol and magnolol, but not always limited thereto.

The cancer herein is preferably selected from the group consisting of colorectal cancer, stomach cancer, prostatic cancer, breast cancer, kidney cancer, hepatoma, brain tumor, lung cancer, uterine cancer, colon cancer, bladder cancer, blood cancer and pancreatic cancer, but not always limited thereto.

The extracts of Magnolia obovata and active fractions thereof are preferably prepared by the method comprising the following steps:

1) adding water, alcohol or a mixture thereof to the dried fruits or floral buds of Magnolia obovata, followed by extracting the Magnolia obovata extract; and

2) drying the Magnolia obovata extract of step 1) under reduced pressure, followed by column chromatography to give active fractions, but not always limited thereto.

In this method, the Magnolia obovata of step 1) is preferably Magnolia obovata Thunberg and can be either cultivated or purchased.

In this method, the alcohol of step 1) is preferably C₁-C₄ lower alcohol and the lower alcohol herein is preferably ethanol or methanol, but not always limited thereto. Water, alcohol or a mixture thereof is added to the dried fruits or floral buds of Magnolia obovata 2-10 times the weight of the dried fruits or floral buds, and more preferably 3-5 times the weight, but not always limited thereto. The temperature for the extraction is preferably 30-100° C., and more preferably 50-80° C., but not always limited thereto. The extraction time is preferably 1-7 days, and more preferably 4-5 days, but not always limited thereto. After extracting according to the above method, the extract is filtered and concentrated under reduced pressure to give the final extract of Magnolia obovata.

In this method, the column chromatography of step 2) can be performed by using the column filled with a filler selected from the group consisting of silica gel, sephadex, RP-18, polyamide, Toyopearl and XAD resin for the isolation and purification. The column chromatography using a proper filler can be repeated several times. And hexane-ethyl acetate can be used as a solvent, but not always limited thereto.

The present inventors added water, ethanol, methanol and methanol aqueous solution to the dried fruits and floral buds of Magnolia obovata Thunberg respectively, which stood at room temperature for 5 days to give the extracts of Magnolia obovata Thunberg. Each extract was absorbed on silica gel, to which hexane and ethylacetate (8:2) were added for 20-30 minutes, followed by purification to give active fractions.

HPLC was performed to analyze the extracts of Magnolia obovata Thunberg fruits and floral buds and active fractions isolated from the same. As a result, the extracts of Magnolia obovata Thunberg fruits and floral buds and fractions thereof all contained obovatol, honokiol and magnolol (see FIG. 1).

The extracts of Magnolia obovata and active fractions thereof of the present invention are characterized by cancer cell growth inhibitory effect.

To investigate whether or not the extracts of Magnolia obovata Thunberg fruits and floral buds and active fractions thereof could inhibit cancer cell growth, the present inventors treated human lung cancer cells (A549, HEK293 and NCI-H23), breast cancer cells (MDA-MB-231), colon cancer cells (SW620, HCA-7 and HCT116) and prostatic cancer cells (DU145) with the extracts of Magnolia obovata Thunberg and active fractions thereof at different concentrations, and then measured the growth of cancer cells by WST-1 cell proliferation assay kit. Obovatol was also tested likewise. As a result, the extracts of Magnolia obovata and active fractions thereof of the present invention inhibited cancer cell growth approximately 50%, compared with the control (0.1% DMSO), at the concentrations of 10 μg/Ml and more (see Table 1 and FIG. 2).

The present inventors also investigated whether or not the extracts of Magnolia obovata Thunberg fruits and floral buds and active fractions thereof could inhibit metastasis. Particularly, DLD-1 cells, the colon cancer cell line over-expressing PRL-3 playing an important role in cancer cell migration (S. Saha, et al., Science, 294, 1343, 2001), were treated with the extracts of Magnolia obovata and active fractions thereof at different concentrations, and then cancer cell migration was measured by using transwell assay kit. Obovatol was also tested likewise. As a result, the extracts of Magnolia obovata fruits and floral buds and active fractions thereof of the present invention inhibited DLD-1 cell migration at least 50%, compared with the control (0.1% DMSO), at the concentrations of 10 μg/Ml and more (see Table 2 and FIG. 3).

The extracts of Magnolia obovata and active fractions thereof of the present invention characteristically induce apoptosis of cancer cells.

To investigate whether or not the extracts of Magnolia obovata Thunberg fruits and floral buds and active fractions thereof could induce apoptosis of cancer cells, SW620 cells, the colon cancer cell line, were treated with the extracts of Magnolia obovata and active fractions thereof of the present invention at different concentrations. Then, the cells were stained with annexin V-FITC, followed by analysis by flow cytometry. After treating the colon cancer cell line SW620 with the extracts of Magnolia obovata and active fractions thereof of the present invention at different concentrations, protein and RNA were eliminated. DNA was stained with EtBr (ethidium bromide) and photographed under UV. As a result, it was confirmed that the extracts of Magnolia obovata and active fractions thereof of the present invention induced apoptosis of cancer cells at the concentrations of 10 μg/Ml and more.

To confirm in vivo anticancer effect of the extracts of Magnolia obovata and active fractions thereof of the present invention, colon cancer cell line was transplanted in immunodeficient mice, to which the extracts of Magnolia obovata and active fractions thereof of the present invention were orally administered. As a result, it was confirmed that cancer cell growth was inhibited.

These results indicate that the extracts of Magnolia obovata and active fractions thereof of the present invention can induce apoptosis of cancer cells and inhibit cancer cell growth and migration. Therefore, the extracts of Magnolia obovata and active fractions thereof of the present invention can be effectively used as a composition for the prevention and treatment of cancer or a composition for the inhibition of metastasis.

The composition for the prevention and treatment of cancer of the present invention can contain the extracts of Magnolia obovata, active fractions thereof or the mixture of the same and additionally one or more active ingredients having the same or similar functions to the above.

The extracts of Magnolia obovata, active fractions isolated therefrom or the mixture thereof of the present invention can be administered orally or parenterally and be used in general forms of pharmaceutical formulation. The composition of the present invention can be prepared for oral or parenteral administration by mixing with generally used diluents or excipients such as fillers, extenders, binders, wetting agents, disintegrating agents and surfactant. Solid formulations for oral administration are tablets, pills, powders, granules and capsules. These solid formulations are prepared by mixing the pharmaceutical composition of the present invention with one or more suitable excipients such as starch, calcium carbonate, sucrose or lactose, gelatin, etc. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol, gelatin, etc. The composition of the present invention can be. administered parenterally and the parenteral administration includes subcutaneous injection, intravenous injection and intramuscular injection.

The dosage units can contain, for example, 1, 2, 3 or 4 individual doses or ½, ⅓ or ¼ of an individual dose. An individual dose preferably contains the amount of active compound which is administered in one application and which usually corresponds to a whole, ½, ⅓ or ¼ of a daily dose. The effective dosage of the composition of the present invention is 0.01˜10 g/kg per day and preferably 0.1 g˜5 g/kg per day, and administration frequency is 1˜6 times a day. However the effective dosage can be changed according to administration pathway, severity of a disease, gender, weight and age. Therefore, the dosage cannot limit the scope of the present invention by any means.

The composition of the present invention is evaluated to be a safe substance since its estimated LD₅₀ value is much greater than 1 g/kg in rats, which is confirmed by acute toxicity assay with rats tested via oral administration. The composition of the present invention can be administered alone or together with surgical operation, hormone therapy, chemo-therapy and biological regulators to prevent and treat cancer.

The present invention also provides a method for treating cancer containing the step of administering a pharmaceutically effective dose of the extracts of Magnolia otovata or fractions thereof to a subject with cancer.

The present invention also provides a method for preventing cancer containing the step of administering a pharmaceutically effective dose of the extracts of Magnolia obovata or fractions thereof to a subject with cancer.

The present invention also provides a method for inhibiting metastasis containing the step of administering a pharmaceutically effective dose of the extracts of Magnolia obovata or fractions thereof to a subject with cancer.

The present invention also provides a use of the extracts of Magnolia obovata or fractions thereof for the preparation of a composition for the prevention and treatment of cancer or a composition for the inhibition of metastasis.

The present invention also provides a health improving functional food for the prevention and improvement of cancer containing the extracts of Magnolia obovata or fractions thereof as an active ingredient.

In addition, the present invention provides a use of the extracts of Magnolia obovata or fractions thereof for the preparation of a health improving functional food for the prevention and improvement of cancer.

The Magnolia obovata herein is preferably Magnolia obovata Thunberg, but not always limited thereto.

The extracts of Magnolia obovata and active fractions thereof are preferably isolated and purified from the fruits or floral buds of Magnolia obovata, but not always limited thereto.

The extracts of Magnolia obovata and active fractions thereof preferably contain all of obovatol, honokiol and magnolol, but not always limited thereto.

The cancer herein is preferably selected from the group consisting of colorectal cancer, stomach cancer, prostatic cancer, breast cancer, kidney cancer, hepatoma, brain tumor, lung cancer, uterine cancer, colon cancer, bladder cancer, blood cancer and pancreatic cancer, but not always limited thereto.

The extracts of Magnolia obovata, active fractions isolated therefrom or the mixture thereof of the present invention can be used as a food additive. In that case, the extracts of Magnolia obovata, active fractions isolated therefrom or the mixture thereof can be added as they are or as mixed with other food components according to the conventional method. It is preferred to extract Magnolia obovata by using hot water or ethanol and at this time the preferable concentration of ethanol is 50-70%. The mixing ratio of active ingredients can be regulated according to the purpose of use (prevention, health enhancement or treatment). In general, to produce health food or beverages, the extracts of Magnolia obovata, active fractions isolated therefrom or the mixture thereof of the present invention are added preferably by up to 15 weight part and more preferably by up to 10 weight part. However, if long term administration is required for health and hygiene or regulating health condition, the content can be lower than the above but higher content can be accepted as well since the extracts of Magnolia obovata, active fractions isolated therefrom or the mixture thereof of the present invention have been proved to be very safe.

The food herein is not limited. For example, the extracts of Magnolia obovata, active fractions isolated therefrom or the mixture thereof of the present invention can be added to meats, sausages, breads, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent. The preferable content of the natural carbohydrates in the composition of the present invention is 0.01-0.04 g per 100 Ml, and 0.02-0.03 g per 100 Ml is more preferred.

In addition to the ingredients mentioned above, the extracts of Magnolia obovata, active fractions isolated therefrom or the mixture thereof of the present invention can include a variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The extracts of Magnolia obovata, active fractions isolated therefrom or the mixture thereof of the present invention can also include natural fruit juice, fruit beverages and/or fruit flesh addable to vegetable beverages. All the mentioned Ingredients can be added singly or together.

### Advantageous Effect

The composition containing the extracts of Magnolia obovata Thunberg or active fractions thereof of the present invention as an active ingredient inhibit the growth and migration of cancer cells and induce apoptosis of cancer cells, so that they can be effectively used for the production of a composition for the prevention and treatment of cancer, a composition for the inhibition of metastasis and a health improving functional food for the prevention and improvement of cancer.

### DESCRIPTION OF DRAWINGS

The application of the preferred embodiments of the present invention is best understood with reference to the accompanying drawings, wherein:

FIG. 1 is a set of chromatograms illustrating the distributions of honokiol, obovatol and magnolol, major components of Magnolia otovata Thunberg fruits and floral buds (the first graph shows the result of analysis with the mixture comprising standards materials).

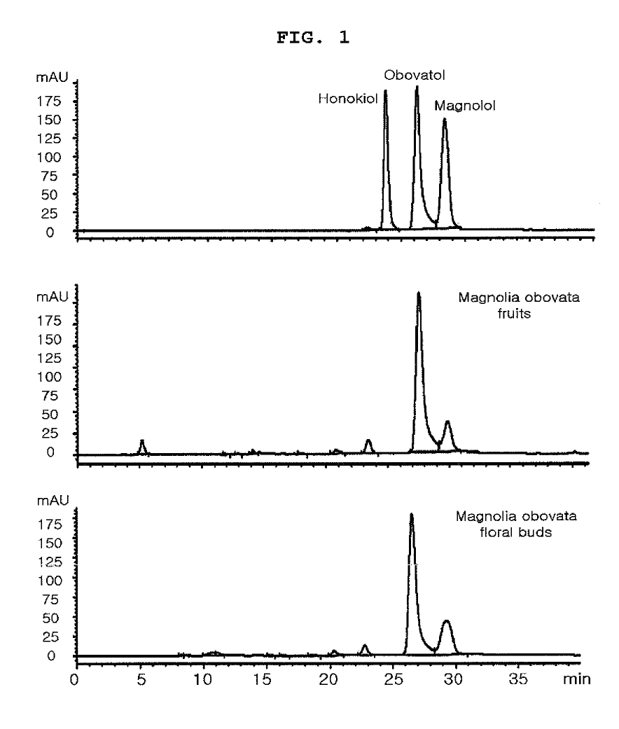

FIG. 2 is a graph illustrating the tumor cell growth inhibitory effect of the methanol extract of Magnolia obovata Thunberg fruits over the concentration.

FIG. 3 is a diagram illustrating the inhibition of DLD-1 cells over-expressing PRL-3 by the methanol extract of Magnolia obovata Thunberg fruits.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of Extracts of Magnolia Obovata

### <1-1> Preparation of Methanol Extract of Magnolia Obovata Fruits

4 L of methanol (22-25° C.) was added to 1 kg of the dried Magnolia obovata Thunberg fruits (picked up directly in Daejeon, Korea: September), which stood at room temperature for 5 days. The extract was filtered with a filter paper and the filtrate was concentrated under reduced pressure by using an evaporator (EYELA, N-1000, Japan) and dried. As a result, 41.5 g of methanol extract of Magnolia obovata Thunberg fruits was obtained.

### <1-2> Preparation of Methanol Extract of Magnolia Obovata Floral Buds

Extraction was performed by the same manner as described in Example <1-1> except that the floral buds (picked up directly in Daejeon, Korea: December) of Magnolia obovata Thunberg were used instead of the fruits of the tree. As a result, 41.0 g of methanol extract of Magnolia obovata Thunberg floral buds was obtained.

### <1-3> Preparation of Ethanol Extract of Magnolia Obovata Fruits

Extraction was performed by the same manner as described in Example <1-1> except that ethanol was used for the extraction instead of methanol. As a result, 40.8 g of ethanol extract of Magnolia obovata Thunberg fruits was obtained.

### <1-4> Preparation of Ethanol Extract of Magnolia Obovata Floral Buds

Extraction was performed by the same manner as described in Example <1-2> except that ethanol was used for the extraction instead of methanol. As a result, 40.4 g of ethanol extract of Magnolia obovata Thunberg floral buds was obtained.

### <1-5> Preparation of 70% Methanol Aqueous Solution Extract of Magnolia Obovata Fruits

Extraction was performed by the same manner as described in Example <1-1> except that 70% methanol aqueous solution was used for the extraction instead of methanol. As a result, 40.5 g of methanol aqueous solution extract of Magnolia obovata Thunberg fruits was obtained.

### <1-6> Preparation of Hot Water Extract of Magnolia Obovata Fruits

Extraction was performed by the same manner as described in Example <11> except that hot water was used for the extraction instead of methanol. As a result, 40.5 g of hot water extract of Magnolia obovata Thunberg fruits was obtained.

### EXAMPLE 2

### Preparation of Active Fractions of the Extracts of Magnolia Obovata

### <2-1> Preparation of Fractions of Methanol Extract of Magnolia Obovata Fruits

The methanol extract of Magnolia obovata Thunberg fruits prepared in Example <1-1> was absorbed on silica gel column, to which hexane and ethyl acetate (8:2) were applied for 20-30 minutes to give 10.5 g of fractions.

### <2-2> Preparation of Fractions of Methanol Extract of Magnolia Obovata Floral Buds

Extraction was performed by the same manner as described in Example <2-1> except that the methanol extract of Magnolia obovata Thunberg floral buds was used instead of the methanol extract of Magnolia obovata Thunberg fruits. As a result, 10.2 g of fractions was obtained.

### <2-3> Preparation of Fractions of Ethanol Extract of Magnolia Obovata Fruits

Extraction was performed by the same manner as described in Example <2-1> except that the ethanol extract of Magnolia obovata Thunberg fruits was used instead of the methanol extract of Magnolia obovata Thunberg fruits. As a result, 9.8 g of fractions was obtained.

### <2-4> Preparation of Fractions of Ethanol Extract of Magnolia Obovata Floral Buds

Extraction was performed by the same manner as described in Example <2-1> except that 10 g of the ethanol extract of Magnolia obovata Thunberg floral buds was used instead of the methanol extract of Magnolia obovata Thunberg fruits. As a result, 9.5 g of fractions was obtained.

### EXAMPLE 3

### Analysis of Components of the Extracts of Magnolia Obovata and Fractions Thereof

The extracts of Magnolia obovata Thunberg fruits and floral buds and fractions thereof obtained in Example 1 and Example 2 proceeded to HPLC for analyzing their components. Conditions for HPLC were as shown in Table 1.

TABLE 1

| Factor | Condition |
|---|---|
| Manufacturer | Hewlett Packard, USA |
| Column | YMC, J□sphere ODS-H80, 250 × 20 mm I.D, S- |
| | 4 uM, 8 nm |
| Moving phase | 80% MeOH: 20% water |
| Moving rate | 4 ml/min |

As a result, it was confirmed that the extracts of Magnolia obovata Thunberg fruits and floral buds and fractions thereof contained all of obovatol, honokiol and magnolol.

### EXPERIMENTAL EXAMPLE 1

### Inhibition of Cancer Cell Growth

Inhibition of human cancer cell growth by the extracts of Magnolia obovata Thunberg and active fractions thereof obtained in Example 1 and Example 2 was investigated by using WST-1 [4-[3-(4-Iodophenyl)-2(4-nitrophenyl)-2H-5-tetrazolio]1,3-benzene disulfonate]. Human lung cancer cells (A549, HEK293 and NCI-H23), breast cancer cells (MDA-MB-231), colon cancer cells (SW620, HCA-7 and HCT116) and prostatic cancer cells (DU145) (ATCC, USA) were cultured in media supplemented with 10% FBS in a 37° C. 5% CO₂ incubator. The cells were detached by using 0.05% trypsin-EDTA. The cells were counted with a hemocytometer and distributed in a 96-well plate at the concentrations of 4×10³ cells/well (A549, MDA-MB-231), 5×10³ cells/well (HEK293, NCI-H23) or 6×10³ cells/well (HCA-7, HCT116, SW620, DU145). The cells were cultured in media supplemented with 10% FBS in a 37° C. 5% CO₂ incubator. 24 hours later, the media were replaced with fresh ones each containing the control (0.1% DMSO), the extracts of Magnolia obovata Thunberg and fractions thereof obtained in Example 1 and Example 2 at different concentrations (the extracts dissolved in DMSO were diluted with media). After 48 hours of the treatment, 10 μl of WST-1 (Roche) was added to each well, followed by culture for 2 hours. OD₄₅₀ was measured with an ELISA reader (Bio-Rad).

TABLE 2

| | Cell survival rate (%) | | | | |
|---|---|---|---|---|---|
| | Concentration (ug/ml) | | | | |
| Experimental group | 0 | 5 | 10 | 15 | 20 |
| Control (0.1% DMSO) | 100 | 100 | 100 | 100 | 100 |
| Extract of Example <1-1> | 100 | 65 | 40 | 32 | 22 |
| Extract of Example <1-2> | 100 | 65 | 40 | 35 | 24 |
| Extract of Example <1-3> | 100 | 66 | 42 | 34 | 22 |
| Extract of Example <1-4> | 100 | 65 | 40 | 38 | 24 |
| Extract of Example <1-5> | 100 | 67 | 44 | 38 | 30 |
| Extract of Example <1-6> | 100 | 68 | 46 | 34 | 32 |
| Fraction of Example <2- | 100 | 63 | 40 | 36 | 28 |
| 1> | | | | | |
| Fraction of Example <2- | 100 | 65 | 42 | 38 | 25 |
| 2> | | | | | |
| Fraction of Example <2- | 100 | 64 | 40 | 36 | 26 |
| 3> | | | | | |
| Fraction of Example <2- | 100 | 65 | 42 | 28 | 30 |
| 4> | | | | | |

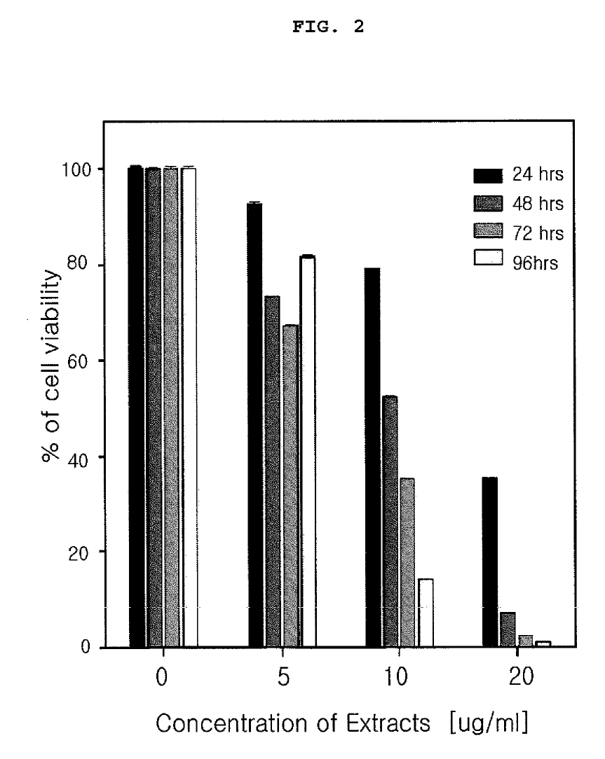

As a result, as shown in Table 1, the extracts of Magnolia obovata Thunberg and fractions thereof inhibited tumor cell growth approximately 50% compared with the control at the concentrations of 10 μg/Ml and more (Table 1 and FIG. 2).

### EXPERIMENTAL EXAMPLE 2

### Inhibition of Tumor Cell Migration

The colon cancer cell line DLD-1 (purchased from ATCC, USA) was cultured and the cells over-expressing PRL-3 (phosphatase of regenerating liver-3) were selected. After removing the culture solution, the cells were washed with PBS. The cells were treated with trypsin-EDTA and cultured to detach the cells. The cells were collected in a falcon tube, to which RPMI(+FBS) was added. The culture solution was eliminated by centrifugation. RPMI(−FBS) was added to the cells, followed by centrifugation twice to eliminate the culture solution. The cells were resuspended in RPMI(−FBS) and counted with a hemocytometer. 8×10⁴ cells/200 ul were added to trans-well (Costar; Cat 3422, USA) of a 24-well plate. 500 ul of RPMI(-FBS) and the control (0.1% DMSO) and the extracts of Magnolia obovata Thunberg and fractions thereof obtained in Example 1 and Example 2 were added to each well of the plate at different concentrations, followed by culture for 12-16 hours. After the culture, the trans-well was placed face down on a hand towel to eliminate the culture solution. 500 ul of crystal violet (5 mg/ml in 20% MeOH) (Sigma; Cat C3886, USA) was added to each well of the 24-well plate, to which trans-well was added, followed by standing for 30 minutes. The stained trans-well was washed with PBS. After smearing in the hand towel, inner side of the membrane was washed with cotton swab to eliminate non-migrated cells, followed by taking pictures and measuring the number of those cells.

TABLE 3

| | Cell migration rate (%) | | | | |
|---|---|---|---|---|---|
| | Concentration (ug/ml) | | | | |
| Experimental group | 0 | 5 | 10 | 15 | 20 |
| Control (0.1% DMSO) | 100 | 100 | 100 | 100 | 100 |
| Extract of Example <1-1> | 100 | 60 | 45 | 36 | 22 |
| Extract of Example <1-2> | 100 | 62 | 48 | 38 | 27 |
| Extract of Example <1-3> | 100 | 64 | 46 | 38 | 28 |
| Extract of Example <1-4> | 100 | 65 | 48 | 36 | 25 |
| Extract of Example <1-5> | 100 | 64 | 50 | 38 | 27 |
| Extract of Example <1-6> | 100 | 70 | 50 | 40 | 30 |
| Fraction of Example <2- | 100 | 60 | 45 | 38 | 26 |
| 1> | | | | | |
| Fraction of Example <2- | 100 | 62 | 48 | 40 | 25 |
| 2> | | | | | |
| Fraction of Example <2- | 100 | 60 | 48 | 38 | 28 |
| 3> | | | | | |
| Fraction of Example <2- | 100 | 64 | 50 | 39 | 28 |
| 4> | | | | | |

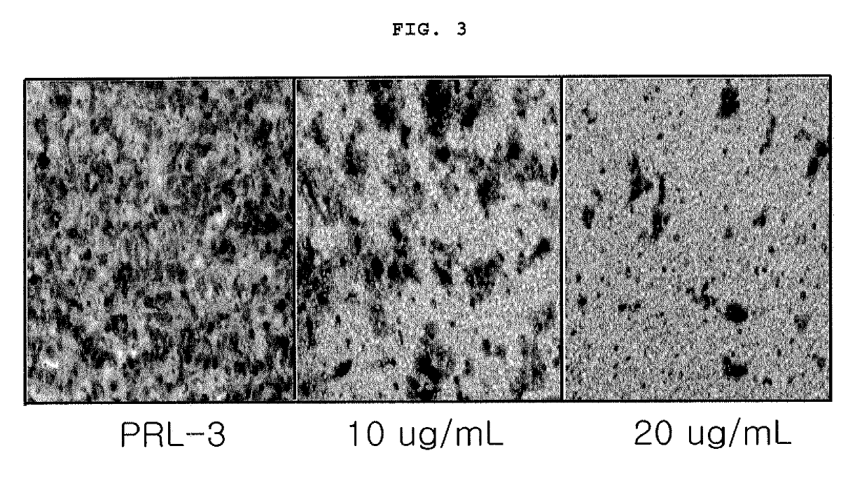

As a result, as shown in Table 2, the extracts of Magnolia obovata Thunberg and fractions thereof inhibited at least 50% of migration of DLD-1 cells over-expressing PRL-3, compared with the control, at the concentrations of 10 μg/Ml and more (Table 2 and FIG. 3).

### EXPERIMENTAL EXAMPLE 3

### Inducement of Apoptosis of Cancer Cells

### <3-1> Investigation of Apoptosis by Flow Cytometry

SW620 cells (human colon cancer cells) were treated with the extracts of Magnolia obovata Thunberg and fractions thereof obtained in Example 1 and Example 2 for 48 hours. The cells were detached by using 0.05% trypsin-EDTA. The cells were recovered by centrifugation, washed twice with PBS (phosphate-buffered saline) and then resuspended in 1× binding buffer (10 mM Hepes/NaOH, pH 7.4, 140 mM NaCl and 2.5 mM CaCl₂) at the concentration of 1×10⁶ cells/Ml. 100 μl of the cell solution was transferred into a 5 Ml culture tube, to which 5 μl of annexin V-FITC (fluorescein isothiocyanate) and 10 μl of PT (propidium iodide) were added, followed by culture at room temperature for 15 minutes under darkness. 400 μl of 1× binding buffer was added to each tube and the number of the stained cells was counted by flow cytometry (Becton Dickinson, USA) to analyze apoptosis (Moussa Alkhalaf, et al., Archives of Medical Research 39, 162-168, 2006).

As a result, the extracts of Magnolia obovata Thunberg and fractions thereof induced apoptosis of cancer cells at the concentrations of 10 μg/Ml and more.

### <3-2> Investigation of Apoptosis by DNA Fragmentation

3×10⁵ colon cancer cells (SW620) were placed in a 10 cm culture vessel, followed by culture for 24 hours. The cells were then treated with 0 1% DMSO (control), the extracts of Magnolia obovata Thunberg obtained in Example 1 or fractions thereof obtained in Example 2 respectively for 48 hours, to which 500 μl of RIPA buffer (50 mM Tris-HCl, pH 7.4, 150 mM NaCl, 1% Triton X100, 0.1% SDS, 5 mM EDTA, 30 mM Na₂HPO₄, 50 mM NaF, 0.5 mM NaVO₄, 2 mM PMSF and 1% aprotinin) was added. The cells were recovered and transferred into a 1.5 Ml tube, followed by shaking culture at 4° C. for 30 minutes. Then, centrifugation was performed at 13,000 rpm for 30 minutes (4° C.). The supernatant was transferred into a 1.5 Ml tube, to which 300 μl of phenol/chloroform (1:1) was added, followed by centrifugation at 4° C. for 20 minutes to eliminate proteins. The supernatant was transferred into a new tube, to which 3 M sodium acetate was added by 10% by the volume. Isopropanol was added thereto by equal volume of the supernatant, followed by centrifugation at 13,000 g at 4° C. for 30 minutes. The precipitate was dissolved in 100 μl of 20 mM Tris-HCl (pH 8.0), to which RNAse (0.1 mg/Ml) was added, followed by culture for one hour at 37° C. to degrade RNA. DNA sample was electrophoresed on 1.5% agarose gel at 100 V, stained with EtBr (ethidium bromide) and then photographed under UV ray.

As a result, the extracts of Magnolia obovata Thunberg and fractions thereof were confirmed to induce apoptosis of cancer cells.

### EXAMPLE 4

### In Vivo Anticancer Effect in Animals

### <4-1> Raising Test Animals and Cancer Cell Line Culture

Female SPF BALB/c nude mice purchased from Charles River, Japan, were used as test animals. They were at 6 weeks and weighed 14-17 g. Before being used for experiments, the test animals were adapted at least one week in a clean animal lab. Temperature of the animal lab was 21±2° C. and humidity was set at 55±5%. Light/dark cycle was automatically regulated as 12/12. The solid feeds for the test animals were radiation sterilized products (Polas International, Seoul, Korea). Drinking water was sterilized by autoclave. Feeds and drinking water were given freely.

For the cancer cell line, human colon cancer cell line SW620 purchased from ATCC, USA and stored at Korean Research Institute of Bioscience and Biotechnology was used. The cells stored in liquid nitrogen were thawed, followed by culture. Particularly, the cancer cell line was cultured in a 37° C. 5% CO₂ incubator (Forma, USA) for a proper time. On the final day of culture, every cell was collected and counted and the final cell concentration was regulated to 1×10⁷ cells/ml by adding saline.

### <4-2> Inhibition of Cancer Tissues in Test Animals

SW620 cancer cells were hypodermically injected in the right armpit in-between the shoulder and the wall of chest by 0.3 ml at the concentration of 3×10⁶ cells/mouse. Samples were orally administered everyday (once a day) from the next day of the cancer cell transplantation (day 1) to the day before autopsy. Particularly, the extracts of Magnolia obovata Thunberg and active fractions thereof of the present invention were administered at the dosage of 10 mg/kg everyday. Weight changes of the test animals were measured three times per week. Tumor volume was measured 7 times from day 5 to day 20 in every test animal. Precisely, the tumor volume was measured by measuring three dimensions of the tumor using Vernier Caliper and then calculated by the following formula:

Tumor volume={Length×Width×Height}/2

On the 20^{th} day from the cancer cell transplantation, the test animals were sacrificed and tumors were separated. They were weighed and photographed with a digital camera and then fixed in liquid nitrogen. Weight changes were observed for 20 days after administering the drug to the SW620 cancer cell transplanted nude mice.

As a result, when the extracts of Magnolia obovata Thunberg and active fractions thereof of the present invention were administered to the animals at the concentration of 10 mg/kg, normal weight increase was observed. In the meantime, the adriamycin (known anticancer agent) administered positive control showed weight loss (13.1% (p<0.001)). The experimental group treated with the extracts of Magnolia obovata Thunberg and active fractions thereof of the present invention demonstrated tumor growth inhibiting effect by them. Particularly, from the 5^{th} day from the SW620 transplantation, the tumor growth of the experimental group treated with the extracts of Magnolia obovata Thunberg and active fractions thereof of the present invention was inhibited 50.0% (P<0.01). In the meantime, the tumor growth of the positive control was inhibited 58.0% (P<0.001).

### EXAMPLE 5

### Acute Toxicity Test in Rats Via Oral Administration

The following experiments were performed to see if the extracts of Magnolia obovata Thunberg and active fractions thereof of the present invention have acute toxicity in rats. 6-week old SPF SD line rats were used in the tests for acute toxicity. The extracts of Magnolia obovata Thunberg and active fractions thereof obtained in Example 1 and Example 2 were suspended in distilled water and orally administered once to 2 rats per group at the dosage of 1 g/kg. Death, clinical symptoms, and weight change in rats were observed, hematological tests and biochemical tests of blood were performed, and any abnormal signs in the gastrointestinal organs of chest and abdomen were checked with the naked eye during autopsy.

The results showed that the test samples did not cause any specific clinical symptoms, weight change, or death in rats. No change was observed in hematological tests, biochemical tests of blood, and autopsy.

The extracts of Magnolia obovata Thunberg and active fractions thereof of the present invention used in this experiment are evaluated to be safe substances since they do not cause any toxic change in rats up to the level of 1 g/kg and their estimated LD₅₀ values are much greater than 1 g/kg in rats.

The Manufacturing Examples of the composition for the present invention are described hereinafter.

### MANUFACTURING EXAMPLE 1

### Preparation of Pharmaceutical Formulations

<1-1> Preparation of powders

| | |
|---|---|
| Extract of Example <1-1> | 2 g |
| Lactose | 1 g |

Powders were prepared by mixing all the above components, which were filled in airtight packs according to the conventional method for preparing powders.

<1-2> preparation of tablets

| | |
|---|---|
| Extract of Example <1-1> | 100 mg |
| Corn starch | 100 mg |
| Lactose | 100 mg |
| Magnesium stearate | 2 mg |

Tablets were prepared by mixing all the above components by the conventional method for preparing tablets.

<1-3> Preparation of capsules

| | |
|---|---|
| Extract of Example <1-1> | 100 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

<1-4> Preparation of pills

| | |
|---|---|
| Extract of Example <1-2> | 1 g |
| Lactose | 1.5 g |
| Glycerin | 1 g |
| Xylitol | 0.5 g |

Pills were prepared by mixing all the above components according to the conventional method for preparing pills. Each pill contained 4 g of the mixture.

<1-5> Preparation of granules

| | |
|---|---|
| Extract of Example <1-2> | 150 mg |
| Soybean extract | 50 mg |
| Glucose | 200 mg |
| Starch | 600 mg |

All the above components were mixed, to which 100 mg of 30% ethanol was added. The mixture was dried at 60° C. and the prepared granules were filled in packs.

### MANUFACTURING EXAMPLE 2

### Preparation of Foods

Foods containing the extracts or fractions of the present invention were prepared as follows.

### <2-1> Preparation of Spices for Cooking

Health enhancing spices for cooking was prepared with 20˜95 weight part of the extract of Example <1-3> according to the conventional method.

### <2-2> Preparation of Tomato Ketchup and Sauce

Health enhancing tomato ketchup or sauce was prepared by mixing 0.2˜1.0 weight part of the extract of Example <1-3> with tomato ketchup or sauce according to the conventional method.

### <2-3> Preparation of Flour Food

0.5˜5.0 weight part of the extract of Example <1-3> was added to the flour. Health enhancing foods such as bread, cake, cookies, crackers and noodles were prepared with the flour mixture according to the conventional method.

### <2-4> Preparation of Soups and Gravies

0.1˜5.0 weight part of the extract of Example <1-3> was added to soups and gravies. Health enhancing meat products, soups and gravies were prepared with this mixture by the conventional method.

### <2-5> Preparation of Ground Beef

Health enhancing ground beef was prepared by mixing 10 weight part of the extract of Example <1-6> with ground beef according to the conventional method.

### <2-6> Preparation of Dairy Products

5˜10 weight part of the extract of Example <1-6> was added to milk. Health enhancing dairy products such as butter and ice cream were prepared with the milk mixture according to the conventional method.

### <2-7> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

The extract of Example <1-6> was concentrated under reduced pressure, spray-dried and pulverized to obtain 60-mesh dry powders.

Sun-Sik was prepared by mixing the dry powders of the grains, seeds and the extract of Example <1-6> according to the below ratio.

Grains (brown rice: 30 weight part, Yulmu: 15 weight part, barley: 20 weight part),

Seeds (wild sesame: 7 weight part, black soybean: 8 weight part, black sesame: 7 weight part),

Dry powders of the compound isolated from the extract of Example <1-6> (3 weight part),

Ganoderma lucidum (0.5 weight part),

Rehmannia glutinosa (0.5 weight part)

<2-8> Preparation of health foods

| Extract of Example <1-3> | 1000 | mg |
|---|---|---|
| Vitamin complex | proper amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Minerals | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition rate for health food. However, the composition rate can be adjusted. The constituents were mixed according to the conventional method for preparing health food and then the composition for health food was prepared according to the conventional method.

### MANUFACTURING EXAMPLE 3

### Preparation of Beverages

<3-1> Preparation of health beverages

| | | |
|---|---|---|
| Extract of Example <1-6> | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | Ml |

The above constituents were mixed according to the conventional method for preparing health beverages. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to regional and national preferences, etc.

### <3-2> Preparation of Vegetable Juice

Health enhancing vegetable juice was prepared by adding 5 g of the extract of Example <1-6> of the present invention to 1,000 Ml of tomato or carrot juice according to the conventional method.

### <3-3> Preparation of Fruit Juice

Health enhancing vegetable juice was prepared by adding 1 g of the extract of Example <1-6> of the present invention to 1,000 Ml of apple or grape juice according to the conventional method.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1-9. (canceled)

10. A method for treating cancer comprising administering a pharmaceutically effective dose of an extract of Magnolia obovata or the active fractions of the extract of Magnolia obovata to a subject with cancer.

11. A method for preventing cancer comprising administering pharmaceutically effective dose of an extract of Magnolia obovata or the active fractions of the extract of Magnolia obovata to a subject.

12-16. (canceled)

17. A method for inhibiting metastasis comprising administering a pharmaceutically effective dose of an extract of Magnolia obovata or the active fractions of the extract of Magnolia obovata to a subject with cancer.

18-24. (canceled)

25. The method of claim 10, wherein the Magnolia obovata is Magnolia obovata Thunberg.

26. The method of claim 10, wherein the extract is extracted from the fruits or floral buds of Magnolia obovata.

27. The method of claim 10, wherein the extract is extracted from Magnolia obovata using water, alcohol, or mixture thereof as a solvent.

28. The method of claim 10, wherein the active fractions of the extract of Magnolia obovata are obtained by absorbing the extract on silica gel column and applying hexane:ethyl acetate (8:2, v/v) thereto for 20-30 minutes.

29. The method of claim 10, wherein the extract of Magnolia obovata or the active fractions thereof contain all of obovatol, honokiol and magnolol.

30. The method of claim 10, wherein the cancer is selected from the group consisting of colorectal cancer, stomach cancer, prostate cancer, breast cancer, kidney cancer, hepatoma, brain tumor, lung cancer, uterine cancer, colon cancer, bladder cancer, blood cancer and pancreatic cancer.

31. The method of claim 10, wherein the cancer is breast cancer, colon cancer or prostate cancer.

32. The method of claim 10, wherein the cancer is colon cancer.

33. The method of claim 11, wherein the Magnolia obovata is Magnolia obovata Thunberg.

34. The method of claim 11, wherein the extract is extracted from the fruits or floral buds of Magnolia obovata.

35. The method of claim 11, wherein the extract is extracted from Magnolia obovata using water, alcohol, or mixture thereof as a solvent.

36. The method of claim 11, wherein the active fractions of the extract of Magnolia obovata are obtained by absorbing the extract on silica gel column and applying hexane:ethyl acetate (8:2, v/v) thereto for 20-30 minutes.

37. The method of claim 11, wherein the extract of Magnolia obovata or the active fractions thereof contain all of obovatol, honokiol and magnolol.

38. The method of claim 11, wherein the cancer is selected from the group consisting of colorectal cancer, stomach cancer, prostate cancer, breast cancer, kidney cancer, hepatoma, brain tumor, lung cancer, uterine cancer, colon cancer, bladder cancer, blood cancer and pancreatic cancer.

39. The method of claim 11, wherein the cancer is breast cancer, colon cancer or prostate cancer.

40. The method of claim 11, wherein the cancer is colon cancer.

41. The method of claim 17, wherein the Magnolia obovata is Magnolia obovata Thunberg.

42. The method of claim 17, wherein the extract is extracted from the fruits or floral buds of Magnolia obovata.

43. The method of claim 17, wherein the extract is extracted from Magnolia obovata using water, alcohol, or mixture thereof as a solvent.

44. The method of claim 17, wherein the active fractions of the extract of Magnolia obovata are obtained by absorbing the extract on silica gel column and applying hexane:ethyl acetate (8:2, v/v) thereto for 20-30 minutes.

45. The method of claim 17, wherein the extract of Magnolia obovata or the active fractions thereof contain all of obovatol, honokiol and magnolol.

46. The method of claim 17, wherein the cancer is selected from the group consisting of colorectal cancer, stomach cancer, prostate cancer, breast cancer, kidney cancer, hepatoma, brain tumor, lung cancer, uterine cancer, colon cancer, bladder cancer, blood cancer and pancreatic cancer.
